(19) United States
(12) Patent Application Publication
(10) Pub. No.: US 2026/0193593 A1
(43) Pub. Date: Jul. 9, 2026

(54) MICROBIAL MICROGRANULE FORMULATION

(71) Applicant: Chr. Hansen A/S, Hoersholm (DK)

(72) Inventors: Soumi BANERJEE, Birkerod (DK); Hannah SCHUENEMANN, Nienburg (DE)

(73) Assignee: Chr. Hansen A/S, Hoersholm (DK)

(21) Appl. No.: 19/129,261

(22) PCT Filed: Nov. 30, 2023
(86) PCT No.: PCT/EP2023/083655

§ 371 Date: May 12, 2025

(30) Foreign Application Priority Data
Nov. 30, 2022 (EP) 22210518.1

(51) Int. Cl. C12N 1/20; C12N 1/02; C12R 1/125

(52) U.S. Cl. CPC C12N 1/20; C12N 1/02; C12R 2001/125

(57) ABSTRACT

This invention relates to formulation aspects of spore forming bacteria. According to a preferred embodiment, the spore forming bacteria are of the genus Bacillus, such as Bacillus subtilis. The invention also relates to a method of manufacturing spore forming bacteria.

### TECHNICAL FIELD

The present invention generally relates to processes of producing microbial microgranule formulation, and particularly microbial microgranule formulation for agricultural applications.

### TECHNICAL BACKGROUND

Wettable powder (WP) is a common product format for both chemical pesticide and microbial agricultural formulations, where active ingredients are formulated with adjuvants (excipients) and dry-blended. However, WPs are plagued with some inherent disadvantages when it comes to end-use. Since dry powders of various particle sizes and densities are mixed, the smaller particles settle at the bottom and the bigger particles remain on the top giving rise to inhomogeneity over time. The powder segregation becomes worse when the formulation undergoes vibrations during transportation. This inhomogeneity leads to dosage variation during application. Moreover, farmers often complain about dustiness of WPs. Fines are almost inevitable for WPs and inhalation of airborne dust coming from co-formulants or active ingredients pose health hazards to end users. This problem is more severe for microbial formulations since spray dried spores get airborne very quickly and give rise to inhalation risk. Thus, there is a need for new product formats that overcomes these challenges and methods for manufacturing these products.

### SUMMARY

Herein is provided an improved formulation of spore forming bacteria and a method of manufacturing said formulation. The specific formulation is called water-dispersible microgranules (WDMG) and is a result of a manufacturing process according to which, mixing co-formulants with a wet bacillus spore concentrate is followed by spray-drying.

Thus, according to a first aspect, a process for producing spore forming bacteria is provided, comprising the steps of a) adding a starter culture of bacterial cells comprising at least one spore forming bacteria strain to growth medium; b) propagating the cells by fermenting the medium for a period of time until the cells sporulate; c) centrifuging the medium to obtain spore concentrate; d) mixing the spore concentrate with co-formulants; e) drying the mix, to obtain dried product; and f) optionally packing the dried product, wherein the spore concentrate and co-formulant are liquids and the mixing step d) is conducted before the drying step e).

In one embodiment, the mixing is performed using a high-shear mixer.

In one embodiment the co-formulant comprises disintegrant, dispersant or antifoam agent.

In a preferred embodiment, the disintegrant is sodium starch glycolate.

In one embodiment, the drying step e) is conducted with a spray dryer.

In one embodiment, the spore forming bacteria are of the genus Bacillus.

In a preferred embodiment, the spore forming bacteria are Bacillus subtilis.

In a particularly preferred embodiment, the spore forming bacteria are Bacillus subtilis and the co-formulant comprises a disintegrant, which is sodium starch glycolate.

According to a second aspect, a product obtainable by the process according to the first aspect is provided.

According to a third aspect, a formulation for spore forming bacteria, comprising spore forming bacteria and co-formulant is provided.

In one embodiment, the spore forming bacteria are of the genus Bacillus.

In a preferred embodiment, the spore forming bacteria are Bacillus subtilis.

In one embodiment, the co-formulant comprises disintegrant, dispersant or antifoam agent.

In a preferred embodiment, the disintegrant is sodium starch glycolate.

In a particularly preferred embodiment, the spore forming bacteria are Bacillus subtilis and the co-formulant comprises a disintegrant, which is sodium starch glycolate.

According to a fourth aspect, a product comprising the formulation according to the third aspect is provided.

According to a fifth aspect, use of a product according to the second aspect or the fourth aspect, in agriculture is provided.

In one embodiment, the use in agriculture is as biological pesticide.

### BRIEF DESCRIPTION OF THE FIGURES

FIG. 1 is a schematic overview of a process line according to a reference embodiment.

FIG. 2 is a schematic overview of a process line according to a embodiment.

FIG. 3 are graphs showing particle size distribution according to different embodiments.

FIG. 4 is a graph comparing particle sizes of different embodiments.

FIG. 5 are photographs showing test results according to an embodiment.

FIGS. 6 to 11 are microscopy images showing various embodiments.

### DETAILED DESCRIPTION

This invention relates to formulation aspects of spore forming bacteria. According to a preferred embodiment, the spore forming bacteria are of the genus Bacillus, such as Bacillus subtilis. The invention also relates to a method of manufacturing spore forming bacteria.

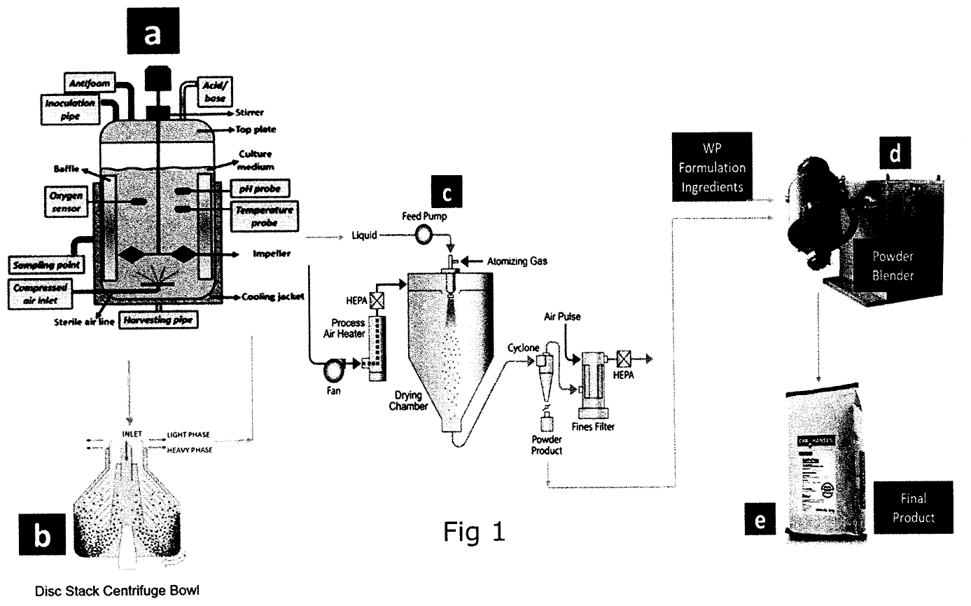

Spore forming bacteria, such as Bacillus subtilis, are fermented based on a starter culture in a fermentation vessel (FIG. 1a), according to standard protocols well know to a person skilled in the art. Spores are formed from vegetative cells. Next, the fermentation media, including cells, is transferred to a centrifuge (FIG. 1b), where spores are concentrated forming spore concentrate and separated from fermentation media supernatant. The spore concentrate is spray dried, using protocols well known to a person skilled in the art, in a spray drier (FIG. 1c). The resulting dry spore powder is subsequently transferred to a powder blender (FIG. 1d) where it is mixed with co-formulant powders to form final wettable powder product, which is packed (FIG. 1e) and shipped.

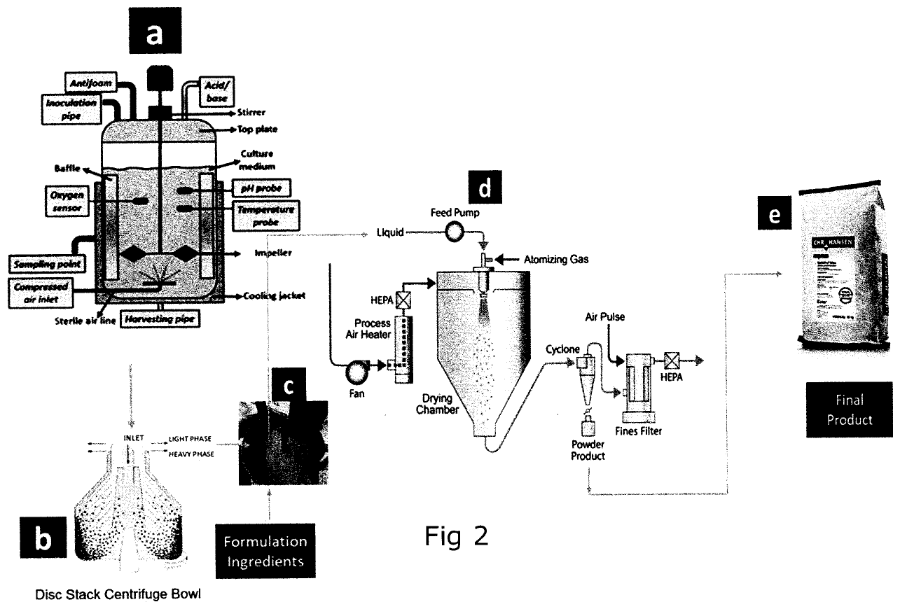

Disclosed herein is a method for manufacturing spore forming bacteria, such as Bacillus subtilis, with several advantages. Spore forming bacteria, such as Bacillus subtilis, are fermented based on a starter culture in a fermentation vessel (FIG. 2a), according to standard protocols well known to a person skilled in the art. Spores are formed from vegetative cells, when the growth cycle enteres a starvation phase. Next, the fermentation media, including spores, is transferred to a centrifuge (FIG. 2b), where spores are concentrated forming spore concentrate and separated from fermentation media supernatant. The spore concentrate is transferred to a mixing tank (FIG. 2c) and mixed with co-formulants. The mixing is preferably done with a high-shear homogenizer. The resulting mix is spray dried, using protocols well known to a person skilled in the art, in a spray drier (FIG. 2d). The resulting water-dispersible microgranules (WDMG) are subsequently packed (FIG. 2e) and shipped.

This method is advantageous because, inter alia, it eliminate the problem of powder segregation during transportation and storage, reduce dustiness and ensures consistent uniformity of dosage.

According to a preferred embodiment, the co-formulants are sodium starch glycolate. Using sodium starch glycolate as co-formulant results in micro-granules, which are more homogenous in size and potency. In a particularly preferred embodiment, the sodium starch glycolate co-formulant is Glycolys® LV. Surprisingly, it was found that Glycolys® LV can very efficiently trap spores and can release the spores after rehydration of the dry powder. Further, the powder formulated with Glycolys® LV is most free-flowing and contains least number of fine particles.

The disclosed method is more safe than currently used methods of manufacturing spore forming bacteria, since the mixing of spores and co-formulants is done in liquid state and thus without generating dust. By using sodium starch glycolate as co-formulant enables this pre-drying mixing, and also improves the properties of the final product.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

TABLE 1
Deposits made at a Depositary institution having acquired the
status of international depositary authority under the Budapest
Treaty on the International Recognition of the Deposit of
Microorganisms for the Purposes of Patent Procedure: Leibniz
Institute DSMZ-German Collection of Microorganisms and Cell
Cultures Inhoffenstr. 7B, 38124 Braunschweig, Germany.

| Strain | Accession No. | Deposit date |
|---|---|---|
| Bacillus subtilis | DSM32324 | 2016 Jun. 8 |

### Formulation Ingredients

The composition of the present invention may additionally comprise cryoprotectants, lyoprotectants, antioxidants, nutrients, fillers, colourants (pigments), excipients, clay, silica, aluminosilicates, fibres, moisture scavengers or mixtures thereof. The composition may be in frozen or freeze-dried form. The composition preferably comprises one or more of formulation ingredients, antioxidants and/or nutrients. Use of standard formulation ingredients are known to a skilled person in the art. Suitable formulation ingredients may include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

### EXAMPLES

### Example 1

### Materials and Methods

Active ingredient in the form of spore concentrate of Bacillus subtilis (DSM32324) was used. The final dry matter (DM) content of the concentrate was 13% (w/w). The active ingredient concentration in the final spray dried powder was 50% (w/w).

Table 2 summarizes the ingredients used in the present example, the composition (dry weight basis) of the formulations tested, the suppliers and functionalities of the ingredients. Three formulations were used, which are named formulation 1, 2 and 3. In addition, a control sample using only spore concentrate without co-formulants was used.

TABLE 2
Names of the ingredients, composition of the formulations, suppliers
of the ingredients and functionalities of the co-formulants

| | Only spore | Formulation 1 | Formulation 2 | Formulation 3 | | |
|---|---|---|---|---|---|---|
| Ingredient | DM (%), w/w | DM (%), w/w | DM (%), w/w | DM (%), w/w | Supplier | Functionality |
| B. subtilis | 100 | 50 | 50 | 50 | Chr. | Active |
| concentrate | | | | | Hansen | ingredient |
| Maltodextrin | 0 | 46.9 | 23.45 | 0 | Roquette | Carrier |
| (DE 12) | | | | | | |
| Glycolys ® | 0 | 0 | 23.45 | 46.9 | Roquette | Disintegrant |
| LV | | | | | | |
| Morwet ® | 0 | 3 | 3 | 3 | Nouryon | Dispersant |
| D425 | | | | | | |
| (Silfoam ® | 0 | 0.1 | 0.1 | 0.1 | Wacker | Antifoam |
| SP 150) | | | | | | agent |

Formulation 1, 2 and 3 have 50% (w/w) spores and same amount (3%, w/w) of sodium salt of naphthalene sulfonate condensate (Morwet® D425), which is a dispersant. The antifoam (Silfoam® SP 150) amount is also kept constant at 0.1% (w/w) in all three formulations. Apart from these ingredients, two carrier materials, e.g. Maltodextrin (DE 12) and Glycolys® LV (sodium starch glycolate) were used. Glycolys® LV is a modified cross-linked corn starch which is known as a disintegrant in tablet formulations. In Formulation 2 the maltodextrin and Glycolys® LV ratio is 1:1. Formulation 1 has no Glycolys® LV and Formulation 3 has no maltodextrin.

### Slurry Preparation

A slurry was prepared for formulation 1, 2 and 3, in a manner well known to a person skilled in the art, by mixing the co-formulants with water using a Ultra-Turrax® (Ultra-Turrax® T50, max 10.000 rpm, Janke & Klunkel IKA Labortechnik) high shear mixer at 10.000 RPM for at least 5 minutes, and if needed up to 30 minutes. Formulation 2 and 3 needed more time to mix because of the presence of Glycolys® LV, which soaks up water and then swells up. Hence, in Formulation 3 extra water was added to attain a viscosity, which can be pumped using the pump of the spray drier. After a homogenized solution is achieved the concentrate containing the active ingredient was added under high shear to attain the final slurry. The high shearing was continued for 10 minutes more after adding the concentrate.

### Spray-Drying of Slurry

For spray drying the pilot spray dryer SD/FSD-4.0 (GEA Niro, Germany) was used with the following parameters:
Chamber inlet flow: 425 kg/h
Inlet temperature of the spray-drier: 190° C.
Outlet temperature: 90° C.
Spray-drying time: 13, 17, 18, and 36 min for Control, Formulation 1, Formulation 2, and Formulation 3, respectively.
Flow rate: 15.37, 17.65, 16.67, and 14.00 kg/h for Control, Formulation 1, Formulation 2, and Formulation 3, respectively.

### CFU Measurement

Colony forming unit (CFU) measurement is a quantitative method where the results are reported as CFU/g. 10 g powder is homogenized by stomaching with 190 g diluent (maximum recovery diluent (MRD)), heated at 80° C. for 10 minutes, cooled and serially diluted in MRD. Appropriate dilutions are then spread on the surface of tryptone soy agar (TSA) plates. After aerobic incubation for 16-24 hours at 37° C. colonies are counted. A summary of the results is found in Table 3.

### Bulk Density

The bulk density is calculated from the mass and corresponding volume of a powder sample. The compression factor is determined with a jolting volumeter apparatus (JEL STAV II, J. Engelsmann AG) wherein 250 strokes ensure the powder compression.

$$Bulk density {ρ}_{B} [ \frac{kg}{{m}^{3}} ] = \frac{Weight m [ g ] * 1000 * 1000}{Volume V [ mL ] * 1000}$$

A summary of the results is found in Table 3.

### Carr Index

Carr Index is a ratio of the tapped bulk density of the powder to the freely settled bulk density of the powder.

$$Carr Index CI [ \% ] = \frac{( tapped bulk density {ρ}_{B , tapped} [ \frac{kg}{{m}^{3}} ] - bulk density {ρ}_{B} [ \frac{kg}{{m}^{3}} ] ) * 100}{bulk density {ρ}_{B} [ \frac{kg}{{m}^{3}} ]}$$

A summary of the results is found in Table 3.

### Flowability

The parameter flowability describes the time needed for 100 g of powder to flow through a funnel. After 10 s a jolting volumeter start to tap the sample. After 180 s the measurement is stopped even if some powder is still within the funnel.

A summary of the results is found in Table 3.

### Moisture Content

The determination of moisture or loss on drying is analyzed in a dry mass balance/oven (HX204 Moisture Analyzer, Mettler Toledo) at 105° C. until constant weight. The moisture is calculated from the weight difference.

A summary of the results is found in Table 3.

TABLE 3
Bulk properties of the spray dried powder of
control sample and formulation 1, 2 and 3.

| | | Formulation | Formulation | Formulation |
|---|---|---|---|---|
| Ingredient | Control | 1 | 2 | 3 |
| CFU/g (B. | 9.7 × 10¹¹ | 4.2 × 10¹¹ | 4.0 × 10¹¹ | 3.7 × 10¹¹ |
| subtilis) | | | | |
| Bulk density | 436 | 488 | 521 | 534 |
| (kg/m³) | | | | |
| Carr Index (%) | 18.2 | 23.3 | 24.6 | 13.6 |
| Flowability (s) | >180 | >180 | 150 | 32 |
| Moisture | 5.1 | 5.3 | 5.5 | 4.6 |
| content (%) | | | | |

### Particle Size Distribution

Particle size distribution of the samples were measured both in dry and wet state by static light scattering (SLS) using Sympatec Helos BR laser diffraction system (Sympatec Inc., Clausthal, Germany) using Rodos (powder) and Quixel (liquid) attachments.

### Dry Particle Size

Particle size distribution of dry powder is measured as such.

### Wet Particle Size

Slurries of different formulations were prepared by adding 1% (w/w) SD powder in water and then particle sizes were measured.

### Wettability

Wettability is measured by adding 5±0.1 g powder in a beaker containing 100±1 ml standard water D (342 ppm hardness (Ca²⁺:Mg²⁺=4:1), pH 6.0-7.0) and noting down the complete wetting time using a stop watch.

### Result and Discussion

Viabilities of the control and formulated spores is given in Table 3. CFU of the control SD powder is the highest, 9.7×10¹¹. For all WDMG the CFU count is of the order of 4×10¹¹ (±0.3×10¹¹), which was as expected due to the dilution with the formulation ingredients.

The bulk density of Formulation 3 is the highest among all, whereas the Carr index is the lowest. This must be due to the presence of the Glycolys® LV. The Carr index is an indicator of compressibility and in-turn the flow behavior of powder and is obtained from the bulk and tapped densities of powder. A powder has an excellent flow if its Carr index is between 5 and 15, a good flow if it's between 16 and 18, a fair flow between 19 and 21 and a poor flow between 22 and 35. As seen in Table 3, the flowability follows: Formulation 3>Only spore>Formulation 1>Formulation 2. The flowability was also measured directly and it also indicates that the best candidate is Formulation 3. This means that the presence of the disintegrant is acting as a flow modifier, i.e. an aid in the formulation. The moisture content of Formulation 3 is also the lowest out of 4.

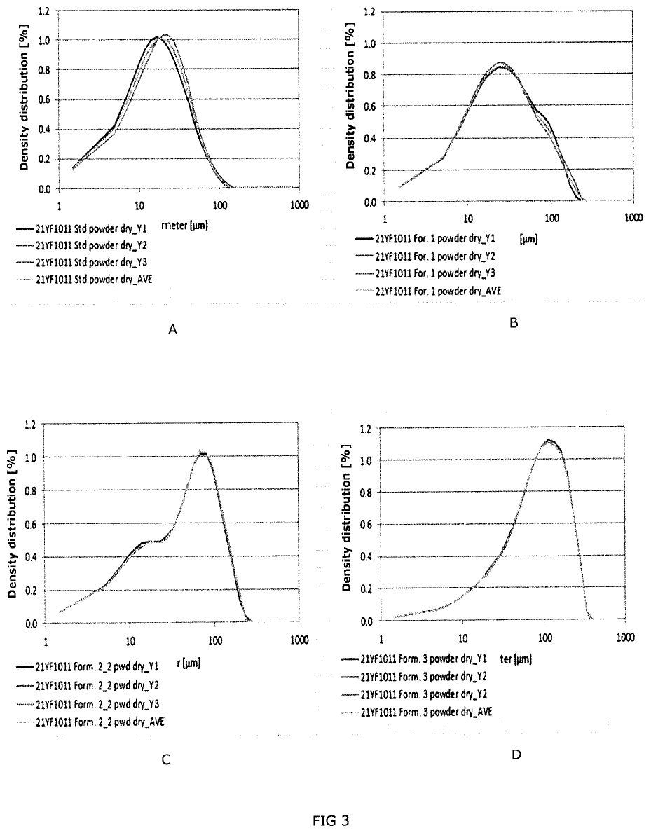

The dry particle size of four SD powders are captured in FIG. 3.

The density distribution (%) versus size clearly show a shift in the distribution as we move from only spore to Formulation 3. The particle size distribution (PSD) for Formulation 3 is monomodal as opposed to the bimodal distribution of Formulation 2. Besides, Formulations 1 and 2 display broader distribution compared to Formulation 3. This indicates that Formulation 3 has less fine particles and more homogeneous particle sizes out of the four. This has huge implications in terms of handling of dry formulations for field applications. Furthermore, absence of fine particles or smaller particles means that internal flow of powder and thereby having segregated mix can also be reduced. So, there will be no separation of smaller particles at the bottom and larger particles on top in the package.

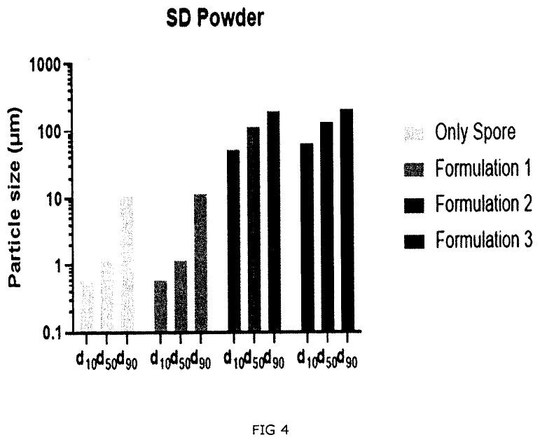

To further delineate the results, values d₁₀, d₅₀ and d₉₀ for all formulations were plotted in FIG. 4. It is clearly seen that for Control (Only Spore) and Formulation 1, d₁₀ and d₅₀ are within 1 and just above 1 μm, respectively. But for Formulations 2 and 3, d₁₀ and d₅₀ shifted towards 100 μm and d₅₀ and doo are close to each other. This indicates uniformity of particle size distribution for Formulations 2 and 3.

The wettability of the microgranules is also of interest and the wetting behavior of the formulations were followed as per the method described in CIPAC MT 53.3.1. FIG. 5 captures the snapshots of the beakers in which the wettability test was performed at the beginning of the test (0s), after 30 s and after 60 s. From the turbidity of the water we clearly see that Formulation 3 renders the water most turbid compared to the other three. This is due to the presence of Glycolys® LV, the disintegrant. Turbidities of formulation 2 and 3 are close since both formulations contain Glycolys® LV. As soon as the formulated microgranules come in contact with water, the disintegrant soaks a large amount of water and swells and the microgranules lose their cohesion and the presence of Morwet® D425 disperses the spores in water. For Formulations 2 and 3, a combined effect of both the disintegrant and Morwet® D425 is seen. However, since Formulation 2 is a mixture of maltodextrin and disintegrant, the effect is less pronounced than that of Formulation 3 where there is no maltodextrin in the formulation.

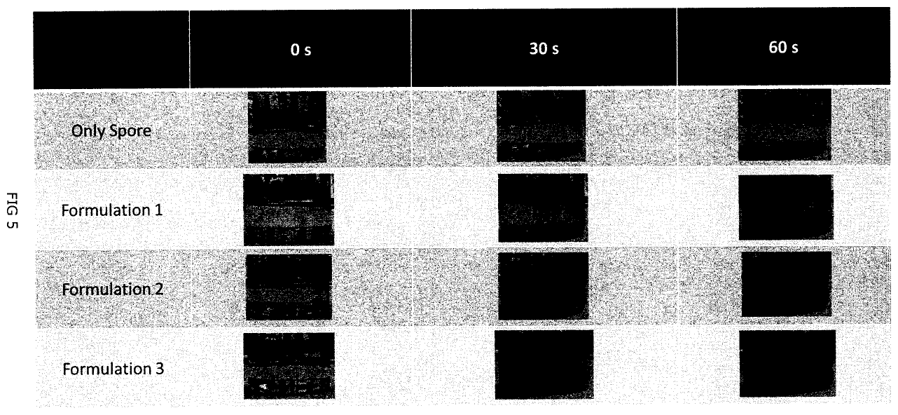

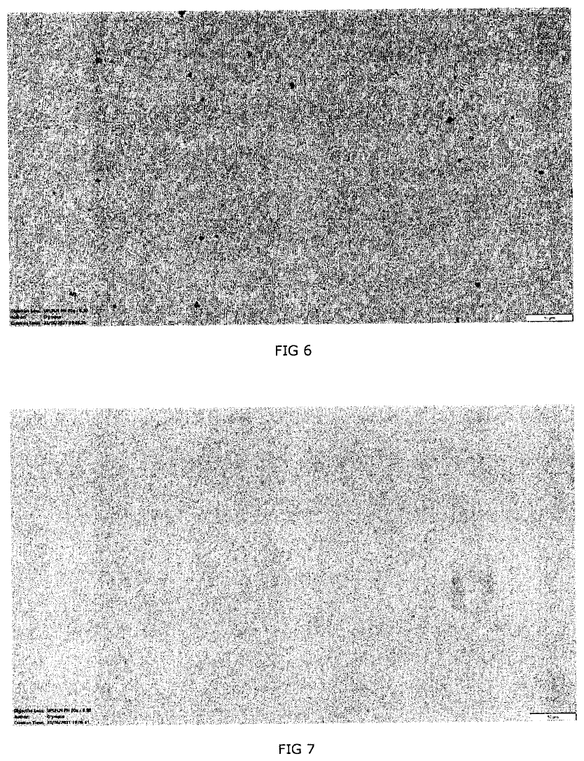

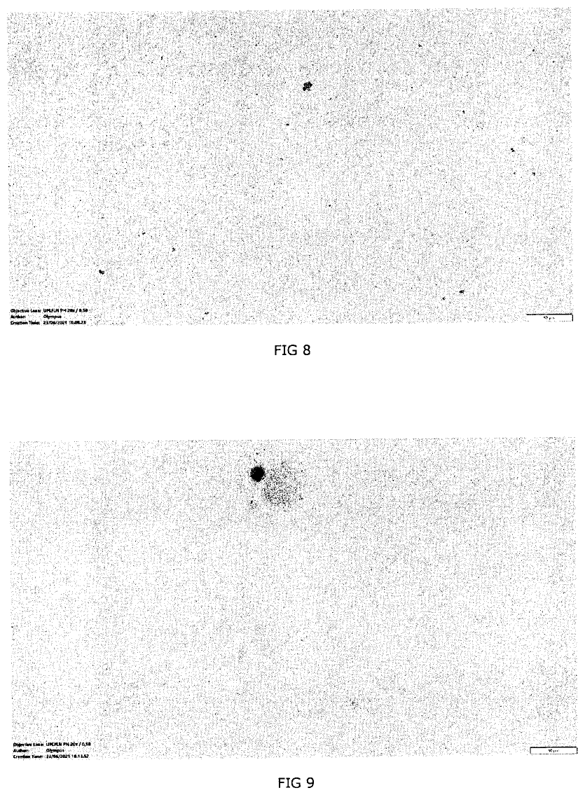

Optical microscopic images of the WDMGs dispersed in water at a concentration of 0.1% (w/w) can be seen from FIGS. 6 to 9. FIG. 6 is a microscopy image of Control, 0.1% dilution in water. FIG. 7 is a microscopy image of formulation 1, 0.1% dilution in water. FIG. 8 is a microscopy image of formulation 2, 0.1% (w/w) dilution in water. FIG. 9 is a microscopy image of formulation 3, 0.1% (w/w) in water. The purple dots are individual spores and the black entities are residues which originate from left-overs from fermentation. The black residues were a concern and it was desired to reduce the residues by the incorporation of dispersants in the formulation.

In FIG. 7 it is clearly seen that the there are less residues in Formulation 2 than Formulation 1.

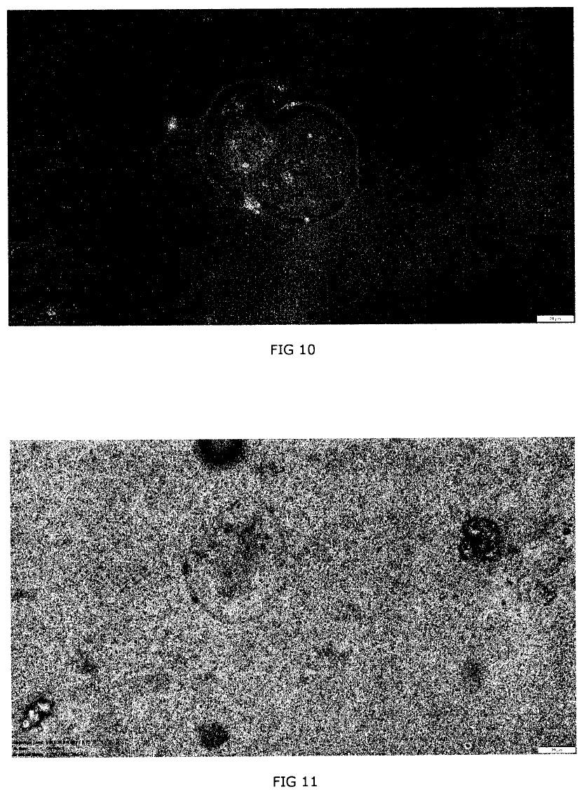

Interestingly, in Formulation 2 well-defined domains are seen, which according to a non-limiting theory of the inventors is a result of the two starches, maltodextrin and Glycolys® LV. These domains are more well-defined in Formulation 3 and it is seen that spores are absorbed by these micro-granules. Interesting enough, much less residue is seen in Formulation 3. Also, in Formulation 3, very round starchy microgranules, with spores embedded, can be seen (FIG. 10, which is a microscopy image of formulation 3, 0.1% (w/w) in water).

In FIG. 11, which again is a microscopy image of Formulation 3, 0.1% (w/w) in water, but at a higher magnification, it is seen that spores are coming out of the granules through a pore on the granule surface.

1. A process for producing spore forming bacteria, said process comprising the steps of:
a) adding a starter culture of bacterial cells comprising at least one spore forming bacteria strain to growth medium;
b) propagating the cells by fermenting the medium for a period of time until the cells sporulate;
c) centrifuging the medium to obtain spore concentrate;
d) mixing the spore concentrate with co-formulants; and
e) drying the mix, to obtain dried product;
wherein the spore concentrate and co-formulant are liquids, and wherein the mixing step d) is conducted before the drying step e).

2. The process according to claim 1, wherein the mixing is performed using a high-shear mixer.

3. The method according to claim 1, wherein the co-formulant comprises disintegrant, dispersant or antifoam agent.

4. The method according to claim 1, wherein the disintegrant is sodium starch glycolate.

5. The method according to claim 1, wherein the drying step e) is conducted with a spray dryer.

6. The method according to claim 1, wherein the spore forming bacteria are of the genus Bacillus.

7. The method according to claim 6, wherein the spore forming bacteria are Bacillus subtilis (DSM32324).

8. A product obtained by the process according to claim 1.

9. A formulation for spore forming bacteria, comprising spore forming bacteria and co-formulant.

10. The formulation according to claim 9, wherein the spore forming bacteria are of the genus Bacillus.

11. The formulation according to claim 10, wherein the spore forming bacteria are Bacillus subtilis (DSM32324).

12. The formulation according to claim 9, wherein the co-formulant comprises disintegrant, dispersant or antifoam agent.

13. The formulation according to claim 12, wherein the disintegrant is sodium starch glycolate.

14-15. (canceled)

16. The formulation according to claim 9, wherein the spore forming bacteria are Bacillus subtilis (DSM32324), and the co-formulant comprises a disintegrant which is sodium starch glycolate.

17. The process according to claim 1, further comprising step f) packing the dried product.
